(19) United States
(12) Patent Application Publication
(10) Pub. No.: US 2026/0144640 A1
(43) Pub. Date: May 28, 2026

(54) ANCHORING UNIT FOR ANCHORING A KNEE PROSTHESIS COMPONENT TO A PATIENT'S LEG BONE, AND PROSTHESIS KIT

(71) Applicant: Aesculap AG, Tuttlingen (DE)

(72) Inventor: Georg Hettich, Freiburg (DE)

(21) Appl. No.: 19/356,273

(22) Filed: Oct. 13, 2025

(30) Foreign Application Priority Data
Oct. 14, 2024 (DE) 10 2024 129 689.5

(51) Int. Cl. A61F 2/30; A61F 2/38; A61L 27/06; A61L 27/22

(52) U.S. Cl. CPC A61F 2/30749; A61F 2/3859; A61F 2/389; A61L 27/06; A61L 27/222; A61F 2002/30004; A61F 2002/30062; A61F 2002/30235; A61F 2002/30331; A61F 2002/30387

(57) ABSTRACT

An anchoring unit is configured for anchoring a knee prosthesis component to a leg bone. The anchoring unit includes an anchoring sleeve having a sleeve wall. The sleeve wall defines a through-opening designed to receive a prosthesis stem. The sleeve wall has at least two wall sections that are formed separately from one another and made of a non-physiologically degradable material. The wall sections are connected to one another by at least one connecting section made of a physiologically degradable material. The anchoring unit can be part of a prosthesis kit.

### CROSS-REFERENCE TO RELATED APPLICATION

This application claims priority under 35 U.S.C. § 119 to German Application No. 10 2024 129 689.5, filed on Oct. 14, 2024, the content of which is incorporated by reference herein in its entirety.

### FIELD

The present disclosure relates to the field of implant technology. In particular, the present disclosure relates to an anchoring unit for anchoring a knee prosthesis component to a patient's leg bone, and to a prosthesis kit.

### BACKGROUND

A knee prosthesis (also called a knee joint prosthesis) is a prosthesis that completely or partially replaces the knee joint. Typically, a knee prosthesis has two knee prosthesis components, viz., a femoral prosthesis component and a tibial prosthesis component, wherein the femoral prosthesis component is implanted in the femur, i.e., the thigh bone, and wherein the tibial prosthesis component is implanted in the tibia, i.e., the shin bone. Such knee prosthesis components typically have a prosthesis stem that carries the actual joint replacement section of the knee prosthesis component.

In the case of bone defects, a sleeve-shaped anchoring unit is often used in addition to the actual knee prosthesis component and serves to anchor the knee prosthesis component to the affected leg bone. Such a sleeve-shaped anchoring unit is also called a “cone.” A cone is usually placed in the metaphyseal region of the leg bone. For this reason, the outer contour of a cone often replicates the metaphyseal region of the tibia or femur bone, thus achieving a good fit. A cone typically has a through-opening designed to accommodate a prosthesis stem of the knee prosthesis component. The knee prosthesis component arranged in this way can then be attached to the cone-for example, using bone cement. The cone facilitates the ingrowth of the leg bone, thereby anchoring the knee prosthesis component to the leg bone.

Such a cone is described, for example, in the published application WO 2024/047127 A1.

In certain situations, it is desirable to again remove an ingrown knee prosthesis component together with the cone, which is also referred to as revision. Typically, the bone cement is removed first, followed by the knee prosthesis component and the cone. A revision is performed, for example, if there is a bacterial infection in the region of the cone or the knee prosthesis component. The advantage of good osteointegration becomes a disadvantage during revision, because the removal of the cone is made more difficult by the ingrowth of the leg bone onto the cone.

### SUMMARY

The present disclosure addresses the object of providing an anchoring unit for a knee prosthesis component that can be removed during a revision in a bone-sparing and time-efficient manner.

A first aspect of the description relates to an anchoring unit for anchoring a knee prosthesis component to a patient's leg bone. The anchoring unit has an anchoring sleeve. In particular, the anchoring unit is formed by the anchoring sleeve, so that the anchoring unit as a whole is sleeve-shaped. However, the anchoring unit may also have one or more further sections in addition to the anchoring sleeve.

The anchoring sleeve has a sleeve wall that defines a through-opening. In particular, the sleeve wall is closed in the circumferential direction such that the sleeve wall completely encloses the through-opening. However, the sleeve wall can also be interrupted in the circumferential direction. The through-opening is designed to accommodate a prosthesis stem of a knee prosthesis component.

The sleeve wall has at least two wall sections which are formed separately from one another and are made of a non-physiologically degradable material. The wall sections are connected to each other by at least one connecting section made of a physiologically degradable material. The connecting section holds the separately formed wall sections together. In particular, the two wall sections are connected to each other only by one or more connecting sections made of a physiologically degradable material.

The inventor has recognized that the underlying object is achieved by an anchoring unit designed as above. After implantation of the anchoring unit and an associated knee prosthesis component, the physiologically degradable material of the connecting section is degraded by the body, so that the connection between the wall sections provided by the connecting section is dissolved. Following implantation, the sleeve wall breaks down into several separate parts, which are preferably held together by bone cement. After removal of the bone cement, the wall sections are then present as such in isolated form, which facilitates the removal of the anchoring unit, especially the now-isolated wall sections.

For the purposes of the disclosure, a “physiologically degradable material” is a material that, after implantation into a leg bone, is degraded by the body in such a way that the structural integrity of a section made of the physiologically degradable material is gradually lost following implantation, in particular over several weeks or several months. Preferably, the physiologically degradable material is completely resorbable. Alternatively, only a component of the physiologically degradable material may be resorbable. For example, the physiologically degradable material may also have a matrix of a resorbable component in which non-resorbable particles are distributed. Even with such a mixed material, the structural integrity is gradually lost as the matrix degrades, making it a physiologically degradable material.

For the purposes of the disclosure, a “non-physiologically degradable material” is a material that does not degrade after implantation into a leg bone, or at most degrades at such a slow rate that the structural integrity of a section made of a non-physiologically degradable material is permanently maintained following implantation.

The sleeve wall has at least two wall sections which are formed separately from one another and are made of a non-physiologically degradable material. More than two such wall sections can also be provided. Preferably, the wall sections are of the same shape.

The wall sections are connected to each other by at least one connecting section made of a physiologically degradable material. There may also be more than one such connecting section present.

Preferably, the at least two wall sections are held together by one or more connecting sections, wherein the wall sections are formed as separate parts. In particular, the separate parts are held together only by one or more connecting sections.

Preferably, the anchoring sleeve is conically tapered. The cross-section of the anchoring sleeve therefore decreases from a first longitudinal end of the anchoring sleeve to a second longitudinal end of the anchoring sleeve. An anchoring sleeve shaped in this way is particularly suitable for placement in the metaphyseal region of a leg bone. The conically tapered anchoring sleeve can be designed either symmetrically or asymmetrically. In a further preferred embodiment, the anchoring sleeve is cylindrical. The anchoring sleeve may also have a conically tapered first longitudinal section and a cylindrical second longitudinal section.

Preferably, the sleeve wall has a structured surface on its outer side, at least in some regions. This can promote the ingrowth of the leg bone to the anchoring unit. The structured surface can, for example, have teeth, grooves, and/or a lattice structure. An example of a suitable lattice structure is commercially available under the registered trademark STRUCTAN® and known as a “STRUCTAN® surface.” Preferably, the wall sections each have the structured surface, at least in some regions.

In some preferred embodiments, it is provided that the wall sections be wall segments of the sleeve wall that are arranged offset from one another. In particular, the wall segments are arranged offset from one another in the circumferential direction of the sleeve wall or in the longitudinal extension of the anchoring sleeve. Wall segments arranged offset from one another have the advantage that they are easily accessible after physiological degradation of the material of the connecting section, and can therefore be easily removed. Preferably, the wall segments are arranged without overlapping, relative to radial viewing directions.

In some preferred embodiments, it is provided that the wall sections be spaced apart from one another, wherein the connecting section is arranged in a space formed between the wall sections. After physiological degradation of the material of the connecting section, a gap remains between the two wall sections. This further facilitates the removal of the two wall sections because their accessibility is improved. Preferably, the two wall sections are designed as wall segments of the sleeve wall that are offset from one another. Preferably, the wall sections and the connecting section are designed such that the outer side of the connecting section is flush with the outer sides of the wall sections. Such an embodiment is facilitated by the provision of the gap and the arrangement of the connecting section in the gap. The outer sides are the sides, facing away from the through-opening, of the sections. Preferably, the connecting section fills the space between the two wall sections. Preferably, the space is gap-shaped. Preferably, the connecting section is arranged only in the space formed between the wall sections. The connecting section therefore does not protrude radially beyond the contour defined by the wall sections.

In some preferred embodiments, it is provided that the wall sections be plate-shaped. In a plate-shaped element, the length and width of the element are significantly larger than its thickness. A plate-shaped element is therefore substantially two-dimensional. Due to the plate-shaped design of the wall sections, the wall sections still effectively anchor the knee prosthesis component to the leg bone even after the connecting section has been degraded. The plate-shaped wall sections are preferably curved. An anchoring sleeve with curved wall sections can be more easily fitted into the metaphyseal region of a leg bone.

In some preferred embodiments, it is provided that the connecting section be rod-shaped. In a rod-shaped element, the length of the element is significantly greater than its width and height. A rod-shaped element is therefore substantially one-dimensional.

In some preferred embodiments, it is provided that the wall sections be arranged one behind the other in the circumferential direction of the anchoring sleeve. The connecting section preferably extends between the wall sections and in the longitudinal extension of the anchoring sleeve.

Preferably, several wall sections and several connecting sections are provided, wherein the wall sections and the connecting sections together form the sleeve wall closed in the circumferential direction, wherein the wall sections are arranged one behind the other in the circumferential direction of the sleeve wall, and wherein a connecting section is always arranged between two adjacent wall sections and connects the two wall sections to one another. Preferably, the wall sections each extend along a circumferential angle interval of at least 30°. The connecting sections preferably each extend along a circumferential angle interval of at most 10°.

In some preferred embodiments, it is provided that at least one of the wall sections be connected to the connecting section by a form-fit connection. This allows a mechanically robust connection between the wall section and the connecting section to be achieved.

In some preferred embodiments, it is provided that at least one of the wall sections have a recess, and that the connecting section project into the recess to form the form-fit connection. The recess may be formed in a circumferentially facing, lateral side of the wall section. Preferably, the recess is filled by the connecting section. Preferably, the recess is elongated-for example, in the form of a groove, a channel, or a furrow.

In some preferred embodiments, it is provided that the recess have an undercut, and that the connecting section engages behind the undercut to form the form-fit connection. Such a design of the form-fit connection is, mechanically, particularly robust. Preferably, the connecting section has a dovetail-shaped projection which projects into the recess and engages behind the undercut.

In some preferred embodiments, it is provided that the physiologically degradable material be designed to be deformable. Consequently, the arrangement of the wall sections relative to each other can be changed at least slightly, viz., by deforming the connecting section. This has the advantage of making it easier to position the anchoring sleeve in the leg bone. Depending upon the shape of the wall sections and the arrangement of the wall sections relative to each other, for example, the length and/or diameter of the anchoring sleeve can be changed by deforming the connecting section or sections. Preferably, the physiologically degradable material is elastically deformable.

The non-physiologically degradable material is preferably rigid. This makes the wall sections mechanically robust; however, they cannot be deformed themselves during implantation of the anchoring sleeve.

In some preferred embodiments, the physiologically degradable material comprises a polymer material. A polymer material is a material that contains macromolecules. The macromolecules are made up of one repeating unit or of several different repeating units.

Preferably, the polymer material is a protein material. The connecting section is thus made of a protein material. Protein materials are particularly suitable both in terms of their mechanical properties and their degradation behavior. The protein material can be a natural, particularly plant or animal, or a synthetic protein material. Preferably, the physiologically degradable material has gelatin as the protein material. Gelatin is particularly suitable due to its good biocompatibility.

Preferably, the polymer material is a polyester material. Particularly preferably, the polyester material comprises poly-L-lactide (PPLLA), poly-lactide-co-glycolide (PLGA), and/or poly-D, L-lactide (PDLLA).

In some preferred embodiments, it is provided that the physiologically degradable material comprises a physiologically degradable ceramic material. Particularly preferably, the physiologically degradable ceramic material comprises hydroxyapatite, alpha-tricalcium phosphate, and/or beta-tricalcium phosphate.

In some preferred embodiments, the physiologically degradable material comprises a physiologically degradable metal or a physiologically degradable metal alloy. Preferred physiologically degradable metals include iron, zinc, and magnesium. Preferred physiologically degradable metal alloys are magnesium-containing metal alloys. Particularly preferably, the physiologically degradable material comprises a magnesium alloy containing zinc and calcium.

Mixtures of the physiologically degradable materials discussed above are also possible. For example, the physiologically degradable material can also be a mixture of a polymer material and a ceramic material.

In some preferred embodiments, the non-physiologically degradable material is a metal or a metal alloy. The wall sections are therefore made of a metal or a metal alloy. Preferably, the non-physiologically degradable material is titanium. Titanium is particularly suitable due to its high rigidity and good biocompatibility. Preferably, the wall sections are manufactured by an additive manufacturing process, particularly preferably by selective laser melting.

In some preferred embodiments, it is provided that the sleeve wall have an inner side, and that at least one securing anchor be formed on the inner side and project from the inner side into the through-opening. As previously mentioned, the prosthesis stem is typically attached to the anchoring unit by bone cement, wherein the bone cement is introduced into the through-opening, in particular between the sleeve wall and the prosthesis stem. The securing anchor is cast with bone cement, thus ensuring a, mechanically, particularly robust fixation. Preferably, each of the wall sections has at least one securing anchor.

A second aspect of the description relates to a prosthesis kit. The prosthesis kit comprises an anchoring unit designed as described above and a knee prosthesis component having a prosthesis stem that can be arranged in the through-opening of the anchoring unit. The prosthesis stem carries the actual joint replacement section of the knee prosthesis component.

With regard to the advantages that can be achieved with the prosthesis kit, reference is made to the statements relating to the anchoring unit in this respect. The features described in connection with the anchoring unit can serve for the further development of the prosthesis kit.

In some preferred embodiments, the prosthesis kit includes bone cement for attaching the knee prosthesis component to the anchoring unit.

### BRIEF DESCRIPTION OF THE DRAWINGS

In the following, the present disclosure will be explained in more detail on the basis of the drawings. In the drawings:

FIG. 1 is a perspectival view of an anchoring unit for anchoring a knee prosthesis component to a leg bone;

FIG. 2 is a sectional view of the anchoring unit with a knee prosthesis component arranged therein; and

FIG. 3 shows a detail of a sleeve wall of the anchoring unit.

### DETAILED DESCRIPTION

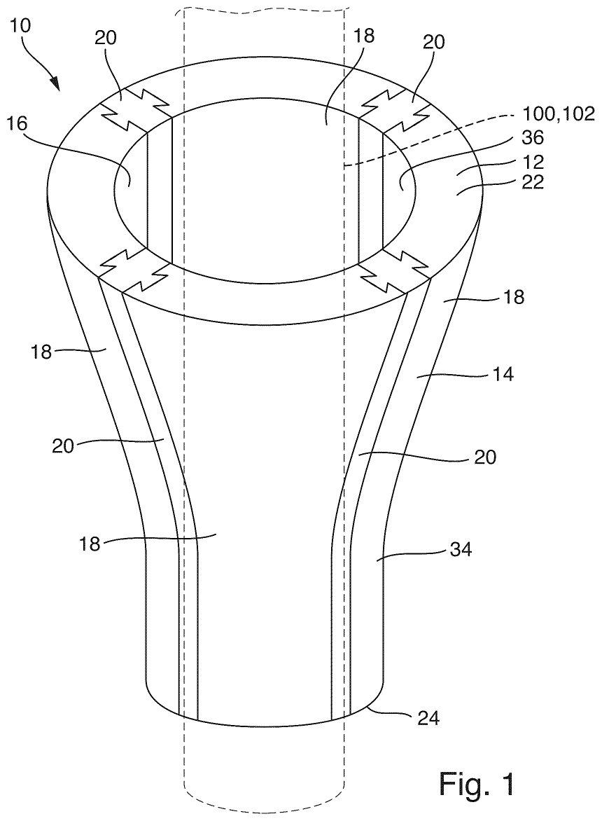

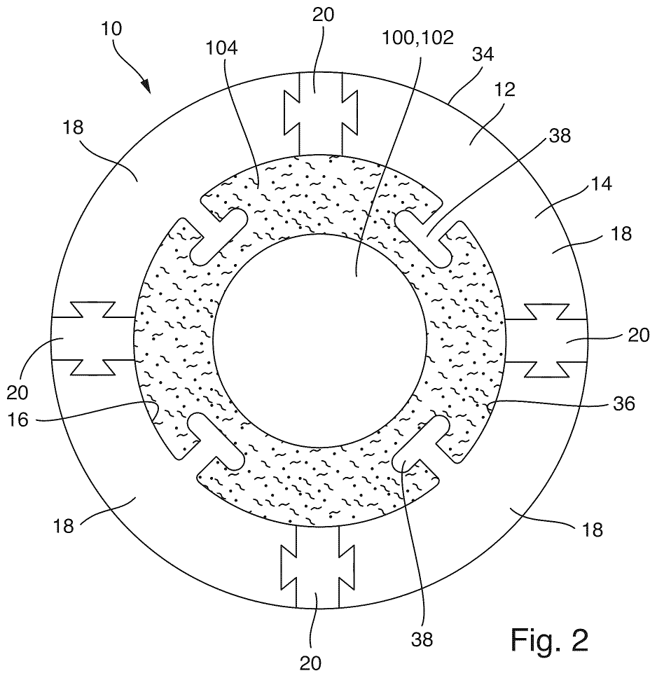

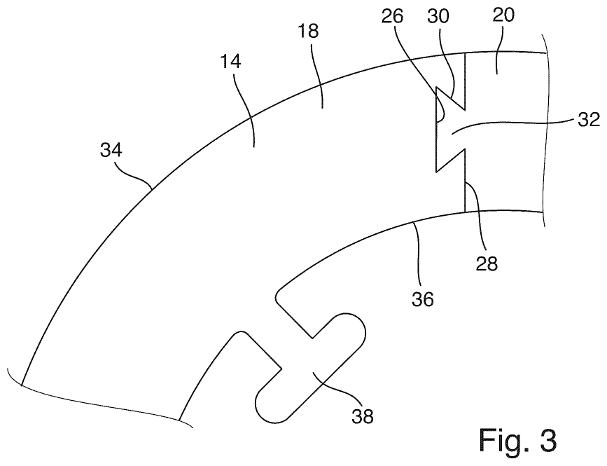

FIG. 1 shows a perspectival view of an anchoring unit 10. FIG. 2 shows a sectional view of the anchoring unit 10, wherein the sectional plane runs perpendicular to the longitudinal center axis of the anchoring unit 10. FIG. 3 shows a detail of a cutout from FIG. 2.

The anchoring unit 10 is intended for anchoring a knee prosthesis component 100 to a patient's leg bone. Depending upon the type of knee prosthesis component 100, the leg bone can be the femur or the tibia.

The anchoring unit 10 has an anchoring sleeve 12. In the present case, the anchoring unit 10 is formed by the anchoring sleeve 12. Alternatively, the anchoring unit 10 may have one or more further, non-sleeve-shaped sections in addition to the anchoring sleeve 12.

The anchoring sleeve 12 has a sleeve wall 14 which defines a through-opening 16. In the present case, the sleeve wall 14 is formed circumferentially, i.e., closed in the circumferential direction of the anchoring sleeve 12. The through-opening 16 is designed to receive a prosthesis stem 102 of the knee prosthesis component 100. A prosthesis stem 102 arranged in this way is indicated by dashed lines in FIG. 1. As can be seen in FIG. 1, the prosthesis stem 102 projects axially through the anchoring sleeve 12. The section, simulating the knee joint, of the knee prosthesis component 100 is carried by the prosthesis stem 102, but is not shown for the sake of simplicity.

During the implantation of the anchoring unit 10 and the knee prosthesis component 100 into a leg bone, the prosthesis stem 102 can be attached to the anchoring sleeve 12 by bone cement 104. The bone cement 104 can fill a gap between the prosthesis stem 102 and the sleeve wall 14, as shown in FIG. 2.

The anchoring sleeve 12 is in this case conically tapered. An anchoring sleeve 12 shaped in this way is particularly suitable for placement in the metaphyseal region of a leg bone. In the present case, the anchoring sleeve 12 has a circular cross-section, as shown in FIG. 2.

The sleeve wall 14 has several wall sections 18 which are formed separately from one another. The wall sections 18 are made of a non-physiologically degradable material. In the exemplary embodiment shown in the figures, exactly four wall sections 18 are provided. However, there may also be a different number of wall sections 18. In the present exemplary embodiment, the wall sections 18 are of the same shape.

The sleeve wall 14 additionally has multiple connecting sections 20. The separately formed wall sections 18 are connected to each other by the connecting sections 20. The connecting sections 20 thus hold the wall sections 18 together. The connecting sections 20 are made of a physiologically degradable material. In the exemplary embodiment shown in the figures, exactly four connecting sections 20 are provided. However, there may also be a different number of connecting sections 20. In the present exemplary embodiment, the connecting sections 20 are of the same shape.

Following the implantation of the anchoring unit 10, the material of the connecting sections 20 is gradually physiologically degraded. Such degradation of the material can, for example, take place over several weeks or several months. As a result, the structural integrity of the connecting sections 20 is lost, and the wall sections 18 are ultimately isolated. If applicable, the isolated wall sections 18 are held together by the bone cement 104. The isolation of the wall sections 18 following implantation has the advantage that the removal of the anchoring unit 10 is facilitated-for example, during a subsequent revision due to infection.

In the exemplary embodiment shown in the figures, the wall sections 18 are wall segments of the sleeve wall 14 arranged offset from one another. The wall sections 18 are arranged one behind the other in the circumferential direction of the anchoring sleeve 12 and each extend along the entire length of the anchoring sleeve 12, i.e., from a first longitudinal end 22 of the anchoring sleeve 12 to a second longitudinal end 24 of the anchoring sleeve 12.

The wall sections 18 are plate-shaped in the present case and each extend along a circumferential angle interval of approximately 80°. Depending upon the number of wall sections 18, other dimensions of the wall sections 18 can also be provided.

As can be seen in the figures, the wall sections 18 are spaced apart from one another, so that, between two adjacent wall sections 18, there is always a gap-shaped space. The connecting sections 20 are rod-shaped. One of the connecting sections 20 is arranged in each of the spaces. In the exemplary embodiment shown in the figures, the connecting sections 20 also extend along the entire length of the anchoring sleeve 12.

The wall sections 18 are connected to the connecting sections 20 in a form-fit. The type of form-fit connection is explained below with reference to FIG. 3. There, an enlarged detail of a wall section 18 and a connecting section 20 is shown.

As can be seen in FIG. 3, the wall section 18 has a recess 26. The recess 26 is formed in a lateral side 28, pointing in the circumferential direction of the sleeve wall 14, of the wall section 18. In the present case, the recess 26 is elongated and extends along the entire length of the lateral side 28. The recess 26 has an undercut 30.

The connecting section 20 projects into the recess 26 and thereby forms the form-fit connection. For this purpose, the connecting section 20 has a dovetail-shaped projection 32 which engages behind the undercut 30. The projection 32 is elongated in the present case and extends along the entire length of the connecting section 20.

As previously mentioned, the wall sections 18 are made of a non-physiologically degradable material. Preferably, the non-physiologically degradable material is a metal or a metal alloy. Particularly preferably, the wall sections 18 are made of titanium, in particular by an additive manufacturing process.

As previously mentioned, the connecting sections 20 are made of a physiologically degradable material. In this exemplary embodiment, the physiologically degradable material is a polymer material. More preferably, the polymer material comprises or consists of gelatin. Gelatin is particularly advantageous due to its biocompatibility, mechanical properties, and degradation behavior under physiological conditions.

However, other physiologically degradable polymer materials are also possible, e.g., polyester materials.

Other preferred physiologically degradable materials are physiologically degradable ceramic materials and physiologically degradable metals or metal alloys.

In the figures, the outer side 34 of the sleeve wall 14, i.e., the side, facing away from the through-opening 16, of the sleeve wall 14, is shown smooth. Deviating from this, it is preferred that the outer side 34 have a structured surface, at least in some regions. This can promote the ingrowth of the leg bone to the anchoring unit 10. The structured surface can, for example, have teeth, grooves, and/or a lattice structure. A suitable lattice structure, as noted above, is a lattice structure that is commercially available under the registered trademark STRUCTAN® and known as a “STRUCTAN® surface.” Particularly preferably, the wall sections 18 each have the structured surface, at least in some regions.

As can be seen in FIGS. 2 and 3, several securing anchors 38 are provided on the inner side 36 of the sleeve wall 14, i.e., on the side, facing the through-opening 16, of the sleeve wall 14, and protrude from the inner side 34 of the sleeve into the through-opening 16. The securing anchors 38 are enclosed by the bone cement 104 when the bone cement 104 is filled in and thereby reinforce the connection between the bone cement 104 and the anchoring unit 10.

In the present exemplary embodiment, each of the wall sections 18 has a securing anchor 38. The securing anchors 38 can extend along the entire length of the wall sections 18. In the present case, the securing anchors 38 extend only along a limited longitudinal section of the wall sections 18 and are therefore not visible in FIG. 1.

1. An anchoring unit for anchoring a knee prosthesis component to a leg bone of a patient, the anchoring unit comprising:
an anchoring sleeve having a sleeve wall, the sleeve wall defining a through-opening configured to receive a prosthesis stem of a knee prosthesis component,
the sleeve wall comprising wall sections that are formed separately from one another and are made of a non-physiologically degradable material, and
the wall sections being connected to one another by at least one connecting section made of a physiologically degradable material.

2. The anchoring unit according to claim 1, wherein the wall sections are wall segments of the sleeve wall arranged offset from one another.

3. The anchoring unit according to claim 1, wherein the wall sections are spaced apart from one another, and the at least one connecting section is arranged in a space formed between the wall sections.

4. The anchoring unit according to claim 1, wherein the wall sections are plate-shaped.

5. The anchoring unit according to claim 1, wherein the at least one connecting section is rod-shaped.

6. The anchoring unit according to claim 1, wherein the wall sections are arranged one behind the other in a circumferential direction of the anchoring sleeve.

7. The anchoring unit according to claim 1, wherein at least one of the wall sections is connected to the at least one connecting section by a form-fit connection.

8. The anchoring unit according to claim 7, wherein at least one of the wall sections has a recess, and the at least one connecting section projects into the recess to form the form-fit connection.

9. The anchoring unit according to claim 8, wherein the recess has an undercut, and the at least one connecting section engages behind the undercut to form the form-fit connection.

10. The anchoring unit according to claim 1, wherein the physiologically degradable material is deformable.

11. The anchoring unit according to claim 10, wherein the physiologically degradable material is elastically deformable.

12. The anchoring unit according to claim 1, wherein the physiologically degradable material comprises a physiologically degradable polymer material, a physiologically degradable ceramic material, a physiologically degradable metal, and/or a physiologically degradable metal alloy.

13. The anchoring unit according to claim 12, wherein the physiologically degradable material comprises a physiologically degradable protein material.

14. The anchoring unit according to claim 12, wherein the physiologically degradable material comprises a physiologically degradable polyester material.

15. The anchoring unit according to claim 1, wherein the non-physiologically degradable material is a metal or a metal alloy.

16. The anchoring unit according to claim 15, wherein the non-physiologically degradable material includes titanium.

17. The anchoring unit according to claim 1, wherein the sleeve wall has an inner side, and at least one securing anchor is formed on the inner side, wherein the at least one securing anchor projects from the inner side into the through-opening.

18. A prosthesis kit comprising:
the anchoring unit according to claim 1; and
a knee prosthesis component comprising a prosthesis stem that is arrangeable in the through-opening of the anchoring unit.

19. The prosthesis kit according to claim 18, wherein the knee prosthesis component comprises a femoral prosthesis component or a tibial prosthesis component.

20. The prosthesis kit according to claim 18, further comprising bone cement for attaching the knee prosthesis component to the anchoring unit.
